(19) United States
(12) Patent Application Publication
(10) Pub. No.: US 2018/0031663 A1
(43) Pub. Date: Feb. 1, 2018

(54) DETECTING NERVE DAMAGE USING DIFFUSION TENSOR IMAGING

(71) Applicant: Children's Medical Center Corporation, Boston, MA (US)

(72) Inventors: David Borsook, Concord, MA (US); Ronald Borra, Boston, MA (US); Paul Serrano, Boston, MA (US); Lino Becerra, Concord, MA (US)

(21) Appl. No.: 15/548,685

(22) PCT Filed: Feb. 3, 2016
(86) PCT No.: PCT/US2016/016331

§ 371 Date: Aug. 3, 2017

Related U.S. Application Data
(60) Provisional application No. 62/111,340, filed on Feb. 3, 2015.

(51) Int. Cl. G01R 33/563; A61B 5/055; A61B 5/00

(52) U.S. Cl. CPC G01R 33/56341; A61B 5/4041; A61B 5/055; A61B 5/7282; A61B 2576/02

(57) ABSTRACT

A method includes: identifying, by a processing circuit, diffusion tensor imaging (DTI) data of a peripheral nerve in a subject that is associated with an area of pain experienced by the subject; determining, by the processing circuit, one or more DTI-derived measurements from the DTI data for each of one or more nerve branches of the peripheral nerve; and detecting, by the processing circuit, potential nerve damage in a particular nerve branch of the one or more nerve branches based on the one or more DTI-derived measurements associated with the particular nerve branch.

### CROSS-REFERENCE TO RELATED APPLICATION

This application is an International Patent Application which claims the benefit of priority under 35 U.S.C. §119(e) to U.S. Provisional Application No. 62/111,340, filed on Feb. 3, 2015 and entitled, “Detecting Nerve Damage Using Diffusion Tensor Imaging”, the entire content of which is incorporated herein by reference.

### TECHNICAL FIELD

The present disclosure relates generally to neuropathic evaluation techniques, and more particularly, to nerve damage detection using diffusion tensor imaging (DTI). The present disclosed subject matter was supported by Federal Grant No. R01NS065051.

### BACKGROUND

Chronic pain affects millions of people throughout the world with the annual economic cost of moderate to severe pain in billions of dollars in the United States alone. A major challenge in chronic pain is that there are currently no objective assays for diagnosis, evaluating or developing effective treatments. One issue is that the disease of chronic pain is not staged in a manner similar to other diseases, where the severity and potential resistance or response to treatment can be evaluated. As a result, because many patients are evaluated well into the course of their disease state—when the disease has already become resistant to effective treatments—developing a treatment plan for suppressing chronic pain can be highly problematic and often varies on a patient-by-patient basis.

Furthermore, many forms of chronic pain evolve following tissue or nerve injury including trauma, surgery, diabetes, post-herpetic neuralgia, small fiber neuropathies, or following seemingly trivial injury such as post phlebotomy or even relatively trivial injury ankle injury. For instance, there are an estimated 10 million ankle injuries in the United States each year, with a high percentage of these being ankle sprains. Stretch injuries associated with ankle sprains can result in neuropathic pain (e.g., nerve traction, hematoma in the epineural sheath, etc.), causing burning pain, electric-sensation pain, tingling, or numbness.

At the ankle, branches of the tibial (medial planter and lateral planter) nerve and fibular (peroneal) nerve are involved. The incidence of nerve damage varies across studies and depends on the grade of sprain. For instance, among patients with Grade II sprains (i.e., moderate sprains with partial tearing of the ligaments), slightly over 25% of patients experienced at least mild tibial and/or fibular nerve damage. Meanwhile, among patients with Grade III sprains (i.e., severe sprains with gross joint instability and possible ligamentous rupture), over 80% of patients injured their tibial and/or fibular nerves. As a result of such nerve damage, neuropathic evolution and devolution may occur over time. That is, nerve damage often causes alterations in nerve pathology, such as nerve fiber loss, fiber regeneration affecting distal and proximal nerves, and the like, in patients with neuropathic pain.

Various techniques exist for assessing nerve pathology, and more specifically, nerve damage, including magnetic resonance imaging (MRI), computer assisted tomography (CAT) (often in conjunction with myelograms), and electromyograms (EMG), among others. In addition, DTI-based techniques have been employed for the assessment of nerve damage. DTI is known in the art for providing useful structural information, often relating to the brain, as well as muscles and other tissues, by measuring the diffusion of water in tissue in order to produce neural tract images. The biological underpinnings for DTI as a useful measure of nerve integrity include the following:
i) nerve injury proinflammatory cytokines may contribute to neuropathic pain and are increased in human sural nerve biopsies of patients with neuropathic pain;
ii) inflammatory markers and cells (e.g., T lymphocytic markers including CD4 and CD8) are present in the nerve epineural and endoneural compartments, distant from the injury implicating a diffuse process;
iii) axonal damage induces a distribution of TNF alpha along the nerve in neuropathic models (e.g., chronic constriction) involving the sciatic nerves; and
iv) neuroinflammatory changes in the peripheral nerve may have effects on glial cells in the spinal cord and brain.

Recently, various improvements to DTI sequences have been made (e.g., increasing the number of DTI encoding directions), which result in improved spatial resolution. However, there is a need for utilizing DTI-based techniques to detect and assess peripheral nerve damage, such as damage occurring in the tibial and fibular nerves, to allow for effective diagnosis and treatment of the same.

### SUMMARY

The present disclosure provides techniques for evaluating the anatomical integrity of peripheral nerves, e.g., the sciatic nerve and its major divisions (the tibial and fibular nerves, for example), the femoral nerve, and so forth, using diffusion tensor imaging (DTI). In particular, high-resolution DTI imaging can be utilized to perform several DTI-derived measurements on a peripheral nerve of a subject, including the various branches of the peripheral nerve. Consequently, measurement of DTI-related changes in peripheral nerves, such as the sciatic nerve, can help to determine whether nerve tracts thereof are affected and, if so, the degree to which damage has been caused.

According to embodiments of the present disclosure, a method includes: identifying, by a processing circuit, diffusion tensor imaging (DTI) data of a peripheral nerve in a subject that is associated with an area of pain experienced by the subject; determining, by the processing circuit, one or more DTI-derived measurements from the DTI data for each of one or more nerve branches of the peripheral nerve; and detecting, by the processing circuit, potential nerve damage in a particular nerve branch of the one or more nerve branches based on the one or more DTI-derived measurements associated with the particular nerve branch.

The method may further include: identifying, by the processing circuit, a first and second nerve branch of the peripheral nerve based on the DTI data, wherein the particular nerve branch corresponds to the first nerve branch or the second nerve branch.

The peripheral nerve may include a sciatic nerve of the subject, the first nerve branch may include a tibial nerve of the subject, and the second nerve branch may include a fibular nerve of the subject.

DTI data acquisition may be centered around a region that is approximately 10 to 15 centimeters above an upper rim of a patella of the subject.

The peripheral nerve may include a femoral nerve of the subject.

The one or more DTI-derived measurements may include one or more of: a fractional anisotropy (FA) measurement, an apparent diffusion coefficient (ADC) measurement, an average diffusivity measurement, and a motor- or sensory-related neuropathic measurement.

The determining of the one or more DTI-derived measurements from the DTI data may include: generating, by the processing circuit, one or more DTI-derived parametric maps based on FA, ADC, or average diffusivity, respectively.

The detecting of the potential nerve damage in the particular nerve branch may include: determining, by the processing circuit, that a FA measurement of the particular nerve branch is below a threshold value.

The method may further include: comparing, by the processing circuit, DTI data for one or more of a tibial nerve and a fibular nerve in a first leg of the subject in which the area of pain resides to DTI data for one or more of a tibial nerve and a fibular nerve in a second leg of the subject that is unaffected by pain.

The detecting of the potential nerve damage in the particular nerve branch may include: determining, by the processing circuit, that either a difference between a FA measurement of the tibial nerve in the first leg and a FA measurement of the tibial nerve in the second leg, or a difference between a FA measurement of the fibular nerve in the first leg and a FA measurement of the fibular nerve in the second leg, exceeds a threshold value.

The method may further include: comparing, by the processing circuit, DTI data for one or more of a tibial nerve and a fibular nerve of the subject to control data for one or more of a healthy tibial nerve and a healthy fibular nerve.

The detecting of the potential nerve damage in the particular nerve branch may include: determining, by the processing circuit, that either a difference between a FA measurement of the tibial nerve and a control FA measurement of the healthy tibial nerve, or a difference between a FA measurement of the fibular nerve and a control FA measurement of the healthy fibular nerve, exceeds a threshold value.

The method may further include: determining, by the processing circuit, a quality level of the DTI data using a fiber tracking technique.

The method may further include: calculating, by the processing circuit, an approximate or actual fiber count of the first and second nerve branches based on an analysis of the DTI data.

The method may further include: performing, by the processing circuit, a fiber-guided region of interest (ROI) placement in the first or second nerve branches based on fiber-tracking guided measurements of FA, ADC, or average diffusivity of the first or second nerve branches

The method may further include: providing, by the processing circuit, data indicative of the detected potential nerve damage to an electronic display.

Furthermore, according to embodiments of the present disclosure, a non-transitory computer readable medium containing program instructions for detecting potential nerve damage includes: program instructions that identify diffusion tensor imaging (DTI) data of a peripheral nerve in a subject that is associated with an area of pain experienced by the subject; program instructions that determine one or more DTI-derived measurements from the DTI data for each of one or more nerve branches of the peripheral nerve; and program instructions that detect potential nerve damage in a particular nerve branch of the one or more nerve branches based on the one or more DTI-derived measurements associated with the particular nerve branch.

### BRIEF DESCRIPTION OF THE DRAWINGS

The embodiments herein may be better understood by referring to the following description in conjunction with the accompanying drawings in which like reference numerals indicate identically or functionally similar elements, of which:

FIG. 1 illustrates an example depiction of DTI imaging of nerve abnormalities in a subject;

FIGS. 2A-2C illustrate example depictions of DTI measurements taken with respect to different subjects for nerves affected by pain; and

FIG. 3 illustrates an example depiction of DTI measurements taken for healthy nerves.

It should be understood that the above-referenced drawings are not necessarily to scale, presenting a somewhat simplified representation of various preferred features illustrative of the basic principles of the disclosure. The specific design features of the present disclosure, including, for example, specific dimensions, orientations, locations, and shapes, will be determined in part by the particular intended application and use environment.

### DETAILED DESCRIPTION

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. The term “coupled” denotes a physical relationship between two components whereby the components are either directly connected to one another or indirectly connected via one or more intermediary components.

It is understood that one or more of the below methods, or aspects thereof, may be executed by at least one processing circuit. The term “processing circuit” may refer to a hardware device that includes various components, such as a memory and a processor. The memory may be configured to store program instructions, and the processor may be configured to execute the program instructions to perform one or more processes which are described further below. Moreover, it is understood that the below methods may be executed by an apparatus comprising the processing circuit, whereby the apparatus is known in the art to be suitable for detecting potential nerve damage based on DTI data associated with one or more nerves of a patient.

Furthermore, the processing circuit of the present disclosure may be embodied as non-transitory computer readable media on a computer readable medium containing executable program instructions executed by a processor, controller or the like. Examples of the computer readable mediums include, but are not limited to, ROM, RAM, compact disc (CD)-ROMs, magnetic tapes, floppy disks, flash drives, smart cards and optical data storage devices. The computer readable recording medium can also be distributed in network coupled computer systems so that the computer readable media is stored and executed in a distributed fashion, e.g., by a telematics server or a Controller Area Network (CAN).

Referring now to embodiments of the present disclosure, the disclosed techniques separately analyze diffusion tensor imaging (DTI) parameters for different branches of a nerve to identify small fiber damage in one of the branches. As is known in the art, DTI is a magnetic resonance imaging (MRI) technique that may be used to map and characterize the three-dimensional diffusion of water as a function of spatial location. As a result, DTI is capable of characterizing microstructural changes or differences with neuropathology and treatment. For instance, DTI is often used to generate estimates of white matter connectivity patterns in the brain from white matter tractography based on the diffusion tensor, which describes the magnitude, the degree of anisotropy, and the orientation of diffusion anisotropy. From the diffusion tensor, diffusion anisotropy measures, such as the fractional anisotropy (FA), can be computed.

In the neural tract images that are produced, each image element (voxel) reflects the best estimate of the rate of water diffusion at that location. To this end, each voxel contains at least one pair of parameters: a rate of diffusion and a preferred direction of diffusion. Based on voxels and the information associated therewith, the intensities at each position in the DTI image are attenuated, according to the strength (b-value) and direction of the magnetic diffusion gradient, as well as on the local microstructure in which the water molecules diffuse. Then, in order to measure a complete diffusion profile, the DTI scans are repeated with varying diffusion gradients, directional vectors, and strengths. The more attenuated the image is at a given position, the greater diffusion there is in the direction of the diffusion gradient.

The diffusion of water in biological tissues, which is primarily caused by random thermal fluctuations, occurs inside, outside, around, and through cellular structures. Cellular membranes hinder the diffusion of water, causing water to take more tortuous paths, thereby decreasing the mean squared displacement. The diffusion tortuosity and corresponding apparent diffusivity may be increased by either cellular swelling or increased cellular density. In other words, water will diffuse more rapidly in a direction aligned with a cellular structure (e.g., muscle, tissue, nerve, etc.), and more slowly as it moves perpendicular to the direction aligned with the structure. Thus, in the case of a nerve bundle, water will diffuse more rapidly in the direction in which the bundle longitudinally extends (i.e., along the length of the nerve bundle), and more slowly as it moves perpendicular to the bundle. Consequently, the diffusion of water within the nerve bundle will be altered by changes in the bundle's microstructure and organization, making DTI-related techniques a powerful tool for characterizing the effects of injury on neural microstructure.

With respect to the present disclosure, high-resolution, DTI-based imaging can be employed slightly above the patella to capture the bifurcation of the sciatic nerve into the tibial and fibular nerves. In doing so, separate DTI-related parameters are captured for the tibial nerve and fibular nerve. For example, the system may determine FA measurements, apparent diffusion coefficient (ADC) measurements, and/or average diffusivity (trace) measurements individually for each nerve in addition to the fiber-count in each individual nerve based on DTI fiber-tracking analysis. The system may then analyze these values, to determine whether there is any potential fiber damage in either nerve branch.

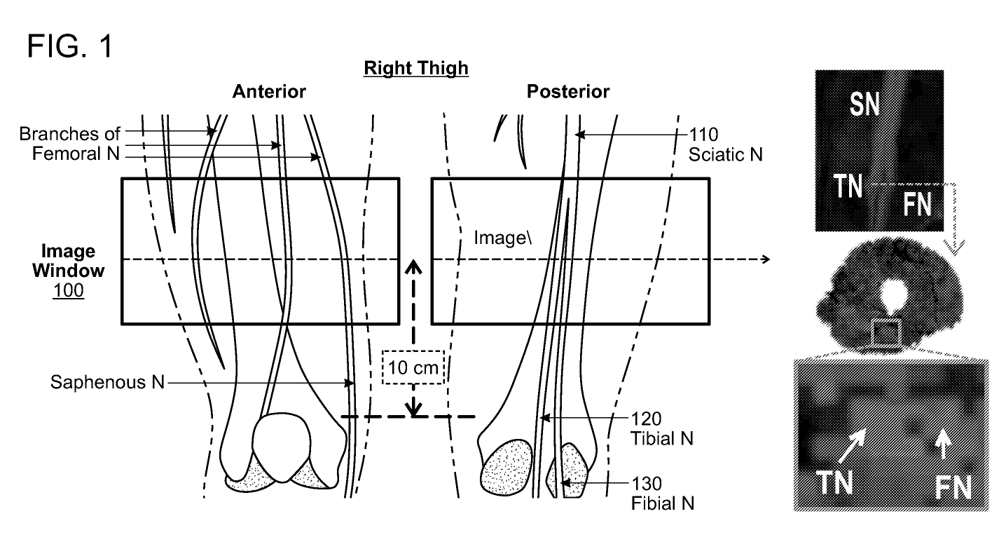

FIG. 1 illustrates an example depiction of DTI imaging of nerve abnormalities in a subject. As generally shown in FIG. 1, DTI data of a peripheral nerve in a subject may be identified, where the peripheral nerve is associated with an area of pain experienced by the subject. The center of the image window 100 (e.g., axial magnetic resonance (MR) slices) may be positioned approximately 10 centimeters above the upper rim of the patella of the subject (e.g., patient), though the center of the axial MR slices may be positioned anywhere from approximately 10 to 15 centimeters above the patella, depending on the leg length of the subject. The DTI data may be acquired using any suitable nerve imaging device, such as a 15-channel Tx/Rx knee coil, as well as any suitable combination of imaging parameters. As a representative, non-limiting example to be understood by a person of ordinary skill in the art, high resolution fat-suppressed 2D turbo spin-echo T2 weighted (2D TSE) images may be acquired for anatomical reference with the following parameters: TR/TE 3630/80 ms, ETL 10, slice thickness 2 mm, inter-slice gap 0.5 mm, matrix 320×224, and FOV 160 mm.

Then, obtained gapless DTI data can be automatically reconstructed, and a fiber tracking-based analysis can be performed to determine/confirm data quality and to reliably identify the sciatic nerve 110 (SN), as well as the tibial nerve 120 (TN) and fibular nerve 130 (FN). That is, the sciatic nerve 110 may represent the peripheral nerve at-issue, and a first and second nerve branch (e.g., tibial and fibular nerve, respectively) of the peripheral nerve may be identified based on the DTI data. Also, a fiber count and/or a fiber-guided ROI placement in the peripheral nerve and the first and second nerve branches may be determined using a fiber tracking technique. Measurements may be separately taken with respect to each of the tibial nerve 120 and fibular nerve 130, in order to detect potential damage in either of the nerve branches, as described in further detail below. Notably, DTI measurements may be taken with respect to any peripheral nerve—not just the sciatic nerve—including, for example, the femoral nerve, nerves in the arm.

Using this data, one or more DTI-derived measurements can be determined for each of one or more nerve branches (e.g., tibial nerve 120 and fibular nerve 130) of the peripheral nerve. To this end, one or more DTI-derived parametric maps can be generated based on FA, ADC, or average diffusivity, respectively. That is, DTI-derived parametric maps associated with FA, ADC, and/or average diffusivity may be generated from a diffusion weighted imaging (DWI) dataset with any suitable combination of imaging parameters. Motor- or sensory-related neuropathic measurements may be utilized, as well. As a representative, non-limiting example to be understood by a person of ordinary skill in the art, the DTI-derived parametric maps may be generated with the following parameters: TR/TE 3900/78 ms, b-values=500 and 800 s/mm2, 12 directions, matrix 134×120 and effective resolution of 1.3×1.3×4 mm Consequently, the FA can be measured for each nerve using the DTI-derived FA maps, averaged over multiple consecutive slices, and then repeated for ADC and/or average diffusivity (trace) maps.

Anatomical T2-weighed imaging data may be used for approximately localizing the sciatic nerve 110. However, regions of interest (ROIs) for analysis can be drawn on b=0 diffusion data, or alternatively can be automatically guided or semi-automatically guided by fiber tracking derived locations of the individual nerve bundles at hand, as it provides a similar level of echo planar imaging (EPI)-related distortion as the parametric FA and ADC maps. The diffusion data may further be post-processed by manually drawing ROIs in the sciatic nerve 110 and transferring them to the FA and ADC maps in multiple adjacent slices (e.g., three above and three below the center slice), after which the average FA and ADC values can be recorded.

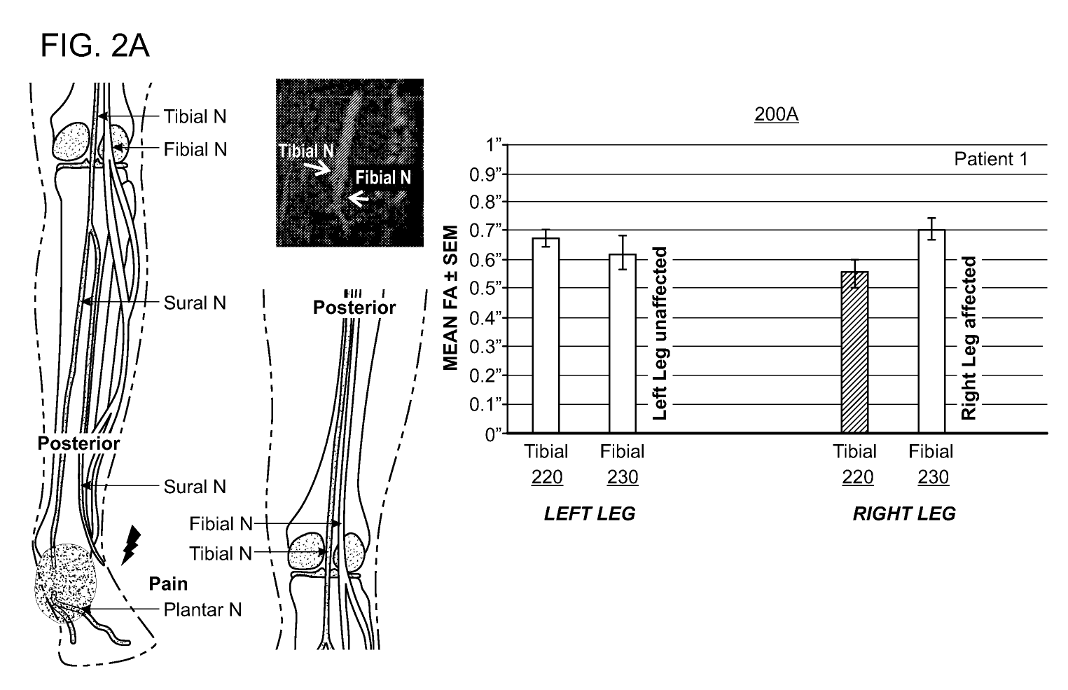

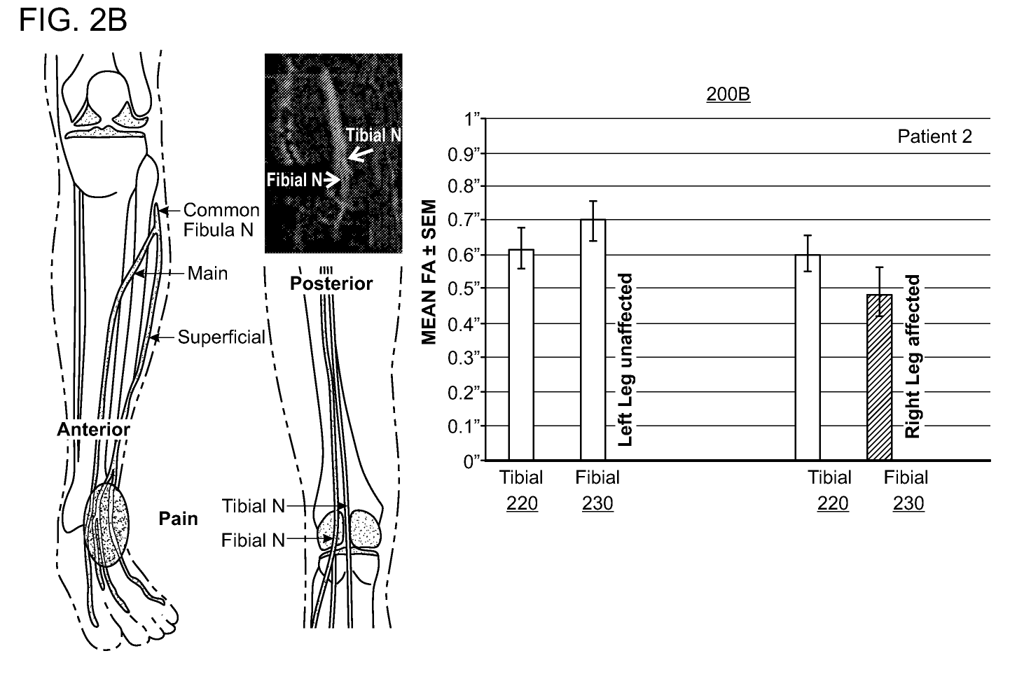

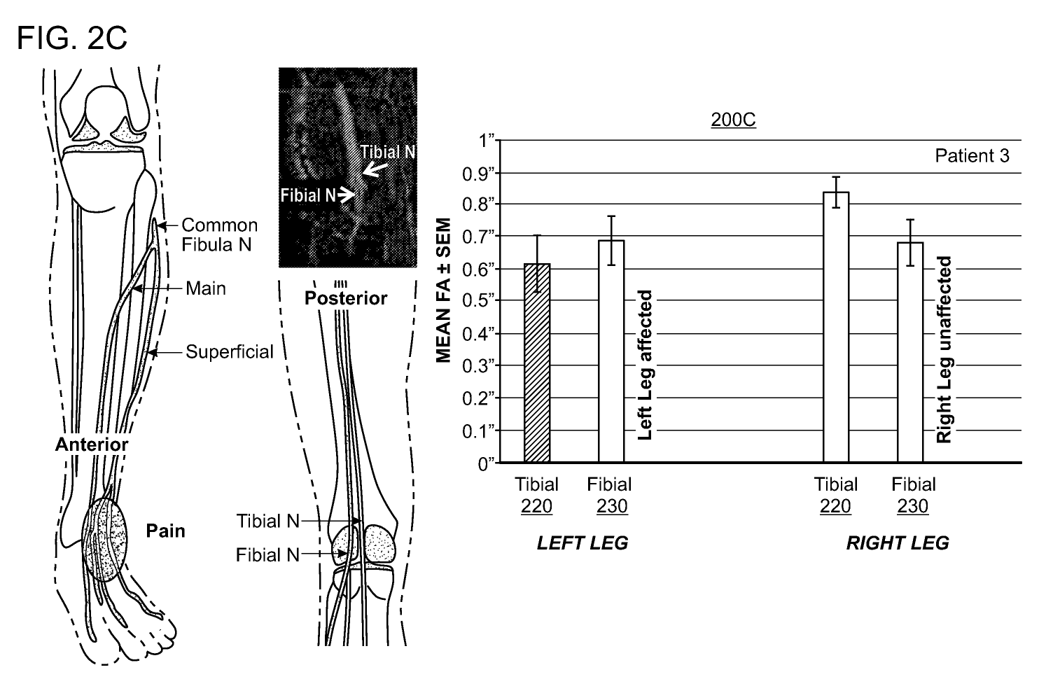

FIGS. 2A-2C illustrate example depictions of DTI measurements taken with respect to different subjects for nerves affected by pain. As generally shown in FIGS. 2A-2C, DTI data can be obtained in three patients (i.e., Patient 1, Patient 2, Patient 3) experiencing neuropathic pain in the area shown in the circle for the tibial and fibular nerves. As similarly shown in FIG. 1, DTI measurements in the thigh region capture and differentiate the fibular nerve 130 from the tibial nerve 120 just below the bifurcation of the sciatic nerve 110, as noted in the DTI fiber-tracking image. Notably, graphs 200A, 200B, and 200C depict FA measurements (from 0 to 1) of the tibial nerve 220 and fibular nerve 230 in the left and right legs of each subject. The FA measurements, as is known in the art, describe the degree of anisotropy of the water diffusion process with a value of 0 reflecting unrestricted (or equally restricted) diffusion in all directions, and a value of 1 reflecting diffusion occurring only along a single axis. Therefore, FA is often used in brain DTI as a measure of fiber density, axonal diameter, and myelination.

In FIG. 2A, the FA graph 200A depicts the tibial nerve 220 in the right leg of Patient 1 having an abnormally low FA of less than 0.6, which is more than 0.1 less than the tibial nerve 220 in the left leg. This disparity is indicative of potential nerve damage in the right tibial nerve 220. Further, in FIG. 2B, the FA graph 200B depicts the fibular nerve 230 in the right leg of Patient 2 having an abnormally low FA of less than 0.5, which is more than 0.2 less than the fibular nerve 230 in the left leg. This disparity is indicative of potential nerve damage in the right fibular nerve 230. Further, in FIG. 2C, the FA graph 200C depicts the tibial nerve 220 in the left leg of Patient 2 having an abnormally low FA of slightly higher than 0.6, which is more than 0.1 less than the tibial nerve 220 in the right leg. This disparity is indicative of potential nerve damage in the left tibial nerve 220. Notably, while a significant decrease in observed FA, as compared to the intact nerve on the unaffected leg, indicates potential neuropathic pain for the particular nerve, there is substantially no difference in observed FA between the tibial and fibular nerve in the opposite, unaffected leg. Accordingly, potential nerve damage in a particular nerve branch may be detected based on one or more DTI-derived measurements (e.g., FA, ADC, average diffusivity, etc.) associated with the particular nerve branch.

The detecting of potential nerve damage in a particular nerve branch (e.g., tibial or fibular nerves) may be performed in a variety of ways. For example, a threshold FA value may be set based on a desired degree of nerve injury. Then, it may be determined whether the FA measurement of the particular nerve branch is below the threshold value. For example, as illustrated in FIG. 2A, if the threshold FA value is set to 0.6, since the FA measurement of the tibial nerve 220 falls below the threshold of 0.6, it may be determined that potential nerve damage exists in the tibial nerve.

Additionally, DTI data for one or more of a tibial nerve and a fibular nerve in a first leg of the subject in which the area of pain resides (e.g., right leg) may be compared to DTI data for one or more of a tibial nerve and a fibular nerve in a second leg of the subject that is unaffected by pain (e.g., left leg). Based on this comparison, the processing circuit may determine whether either a difference between a FA measurement of the tibial nerve in the first leg and a FA measurement of the tibial nerve in the second leg, or a difference between a FA measurement of the fibular nerve in the first leg and a FA measurement of the fibular nerve in the second leg, exceeds a threshold value. For example, as illustrated in FIG. 2B, if the threshold value is set to 0.1, since the difference in FA of the fibular nerve 230 in the (affected) right leg and the FA of the fibular nerve 230 in the (unaffected) left leg exceeds the threshold of 0.1, it may be determined that potential nerve damage exists in the right fibular nerve.

Additionally, DTI data for one or more of a tibial nerve and a fibular nerve of the subject may be compared to control data for one or more of a healthy tibial nerve and a healthy fibular nerve. Based on this comparison, the processing circuit may determine whether either a difference between a FA measurement of the tibial nerve and a control FA measurement of the healthy tibial nerve, or a difference between a FA measurement of the fibular nerve and a control FA measurement of the healthy fibular nerve, exceeds a threshold value. Thus, one or more nerves may be assessed for damage by comparing the FA measurements thereof to FA measurements of a control subject.

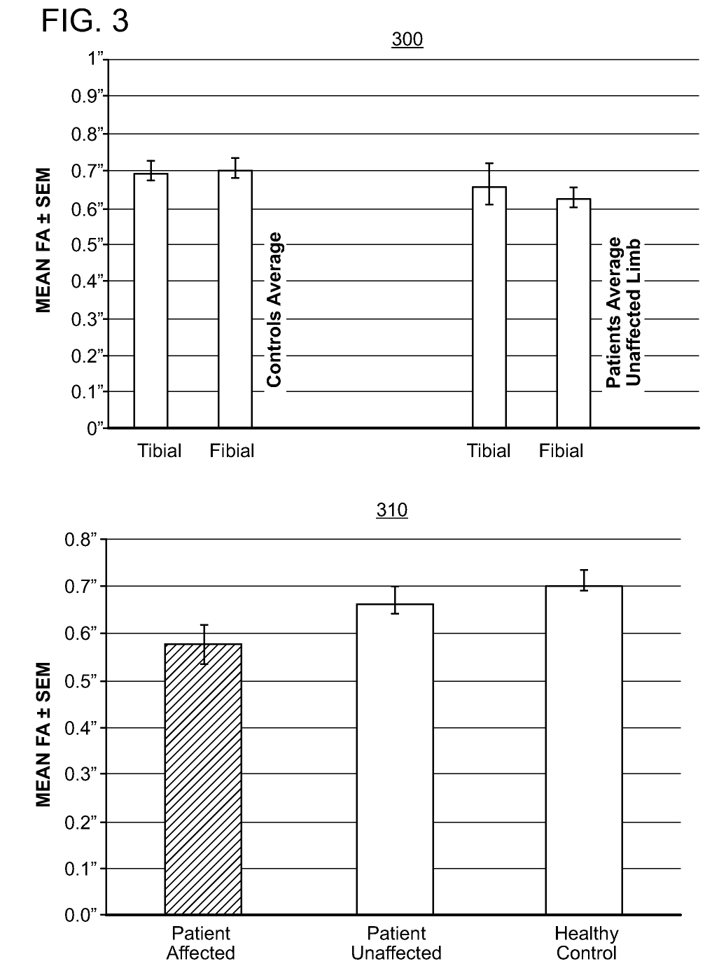

For the sake of comparison, FIG. 3 illustrates an example depiction of DTI measurements taken for healthy nerves. As generally shown in FIG. 3, the graph 300 demonstrates a comparison of FA measurements in a patient's unaffected (i.e., without pain) tibial and fibular nerves against the same nerves in a healthy control model (averages include both the right and left leg). Similarly, the graph 310 demonstrates a comparison of FA measurements in combined affected (i.e., with pain) nerve changes, against matching nerves on the opposite/unaffected side, and further against healthy control averages both legs. Notably, although FA measurements are depicted in FIGS. 2 and 3, any suitable DTI-derived measurement from the acquired DTI data may be employed for the purposes of detecting potential nerve damage (e.g., ADC, average diffusivity, and the like).

Accordingly, the present disclosure is based on the premise that alterations in nerve pathology (e.g., nerve fiber loss, fiber regeneration affecting distal and proximal nerves, and so forth) can occur in patients with neuropathic pain. However, in the case of peripheral nerves, and specifically in the case of spontaneous chronic pain predominantly affecting unmyelinated C-fiber and thinly myelinated Ad fibers, a decreased FA-value reflects a decrease or other change in these fibers, as explained in detail above. Thus, the techniques described herein provide the ability to measure and analyze data reflecting changes in peripheral nerve integrity, thereby allowing for effective treatment plans that counteract the damaged nerves to be developed in a consistent and timely manner.

Neurologic injuries that occur related to regional anesthesia and pain medicine treatment, as a small number of those procedures results in serious persisting deficits of motor or sensory performance, or in the generation of pain. In case of a potential injury, few, if any, methods to objectively quantify nerve integrity and possible response to therapy currently exist, greatly impairing an evidence-based medicine approach for peripheral nerve injury. Therefore, there is a great need for non-invasive methods with a high sensitivity that allow for the assessment of peripheral nerve integrity, such as the proposed DTI-based method. Because this method potentially allows for frequent monitoring of nerve fiber damage, it can be used to evaluate the effectiveness of novel pharmacological treatments, such as those focused on producing an anti-inflammatory response to promote recovery in peripheral nerve damage, as well as providing information on possible optimal dosing schemes in the individual patients. Furthermore, as these pharmacological treatments are not without side-effects, unnecessary treatment in patients not responding over a certain amount of time, as detected by DTI based measurements, could be potentially prevented.

Moreover, current objective measures for nerve alteration often involve painful EMG or nerve biopsy. While the latter is the most objective finding, the presently disclosed approach allows for a non-invasive, non-painful, relatively routine use of MRI imaging. In particular, use of the presently disclosed approach could:
i) allow for evidence of alteration in the nerve that provides an objective correlate of the disease;
ii) be used to define efficacy of treatment (e.g., current clinical approaches, including physical therapy, medication, rest, etc.);
iii) provide a readout for pharmaceutical trials of new drugs for treating chronic pain; and
iv) be used for other diseases where the peripheral nerve is affected, including, for example, amyotrophic lateral sclerosis (ALS), etc.

While there have been shown and described illustrative embodiments that provide for detecting nerve damage using DTI-based techniques, it is to be understood that various other adaptations and modifications may be made within the spirit and scope of the embodiments herein. For example, the embodiments have been primarily shown and described herein with relation to the sciatic nerve, as well as the tibial and fibular nerves. However, the embodiments in their broader sense are not as limited. Rather, the DTI-based detection techniques may, in fact, be used with any nerve in the peripheral nervous system (PNS). In addition, the embodiments have been primarily shown and described herein with relation to one or more DTI-derived measurements including, primarily, FA measurements. However, the embodiments in their broader sense are not as limited, and FA, as well as ADC and average diffusivity, are depicted herein for demonstration purposes only and should not be treated as limiting the disclosed embodiments, since any suitable measurement deriving from acquired DTI data may be utilized within the scope of the present disclosure.

The foregoing description has been directed to embodiments of the present disclosure. It will be apparent, however, that other variations and modifications may be made to the described embodiments, with the attainment of some or all of their advantages. Accordingly, this description is to be taken only by way of example and not to otherwise limit the scope of the embodiments herein. Therefore, it is the object of the appended claims to cover all such variations and modifications as come within the true spirit and scope of the embodiments herein.

What is claimed is:

1. A method comprising:
identifying, by a processing circuit, diffusion tensor imaging (DTI) data of a peripheral nerve in a subject that is associated with an area of pain experienced by the subject;
determining, by the processing circuit, one or more DTI-derived measurements from the DTI data for each of one or more nerve branches of the peripheral nerve; and
detecting, by the processing circuit, potential nerve damage in a particular nerve branch of the one or more nerve branches based on the one or more DTI-derived measurements associated with the particular nerve branch.

2. The method of claim 1, further comprising:
identifying, by the processing circuit, a first and second nerve branch of the peripheral nerve based on the DTI data,
wherein the particular nerve branch corresponds to the first nerve branch or the second nerve branch.

3. The method of claim 2, wherein the peripheral nerve includes a sciatic nerve of the subject, the first nerve branch includes a tibial nerve of the subject, and the second nerve branch includes a fibular nerve of the subject.

4. The method of claim 3, wherein DTI data acquisition is centered around a region that is approximately 10 to 15 centimeters above an upper rim of a patella of the subject.

5. The method of claim 1, wherein the peripheral nerve includes a femoral nerve of the subject.

6. The method of claim 1, wherein the one or more DTI-derived measurements include one or more of: a fractional anisotropy (FA) measurement, an apparent diffusion coefficient (ADC) measurement, an average diffusivity measurement, and a motor- or sensory-related neuropathic measurement.

7. The method of claim 1, wherein the determining of the one or more DTI-derived measurements from the DTI data comprises:
generating, by the processing circuit, one or more DTI-derived parametric maps based on FA, ADC, or average diffusivity, respectively.

8. The method of claim 1, wherein the detecting of the potential nerve damage in the particular nerve branch comprises:
determining, by the processing circuit, that a FA measurement of the particular nerve branch is below a threshold value.

9. The method of claim 1, further comprising:
comparing, by the processing circuit, DTI data for one or more of a tibial nerve and a fibular nerve in a first leg of the subject in which the area of pain resides to DTI data for one or more of a tibial nerve and a fibular nerve in a second leg of the subject that is unaffected by pain.

10. The method of claim 9, wherein the detecting of the potential nerve damage in the particular nerve branch comprises:
determining, by the processing circuit, that either a difference between a FA measurement of the tibial nerve in the first leg and a FA measurement of the tibial nerve in the second leg, or a difference between a FA measurement of the fibular nerve in the first leg and a FA measurement of the fibular nerve in the second leg, exceeds a threshold value.

11. The method of claim 1, further comprising:
comparing, by the processing circuit, DTI data for one or more of a tibial nerve and a fibular nerve of the subject to control data for one or more of a healthy tibial nerve and a healthy fibular nerve.

12. The method of claim 11, wherein the detecting of the potential nerve damage in the particular nerve branch comprises:
determining, by the processing circuit, that either a difference between a FA measurement of the tibial nerve and a control FA measurement of the healthy tibial nerve, or a difference between a FA measurement of the fibular nerve and a control FA measurement of the healthy fibular nerve, exceeds a threshold value.

13. The method of claim 1, further comprising:
determining, by the processing circuit, a quality level of the DTI data using a fiber tracking technique.

14. The method of claim 1, further comprising:
calculating, by the processing circuit, an approximate or actual fiber count of the first and second nerve branches based on an analysis of the DTI data.

15. The method of claim 1, further comprising:
performing, by the processing circuit, a fiber-guided region of interest (ROI) placement in the first or second nerve branches based on fiber-tracking guided measurements of FA, ADC, or average diffusivity of the first or second nerve branches.

16. The method of claim 1, further comprising:
providing, by the processing circuit, data indicative of the detected potential nerve damage to an electronic display.

17. A non-transitory computer readable medium containing program instructions for detecting potential nerve damage, the computer readable medium comprising:
program instructions that identify diffusion tensor imaging (DTI) data of a peripheral nerve in a subject that is associated with an area of pain experienced by the subject;
program instructions that determine one or more DTI-derived measurements from the DTI data for each of one or more nerve branches of the peripheral nerve; and
program instructions that detect potential nerve damage in a particular nerve branch of the one or more nerve branches based on the one or more DTI-derived measurements associated with the particular nerve branch.
